(19) United States
(12) Patent Application Publication
(10) Pub. No.: US 2025/0143951 A1
(43) Pub. Date: May 8, 2025

(54) METHOD AND GUIDANCE DEVICE FOR PROVIDING NON-VISUAL GUIDANCE

(71) Applicant: ASSA ABLOY AB, Stockholm (SE)

(72) Inventors: Kenneth Pernyer, Ängelsberg (SE); Gustav Ryd, Stockholm (SE); Nieves Crasto, Houston, TX (US)

(21) Appl. No.: 18/832,200

(22) PCT Filed: Feb. 10, 2023
(86) PCT No.: PCT/EP2023/053349

§ 371 Date: Jul. 23, 2024

(30) Foreign Application Priority Data
Feb. 11, 2022 (SE) 2250136-5

(51) Int. Cl. A61H 3/06; G01C 21/20; G06V 20/52; G06V 40/10

(52) U.S. Cl. CPC A61H 3/061; G01C 21/206; G06V 20/52; G06V 40/10; A61H 2003/063; A61H 2201/5007; A61H 2201/5092

(57) ABSTRACT

A method for providing non-visual guidance for passing through a door. The method being performed by a guidance device. The method comprises: determining that a person is near the door and that non-visual guidance is to be provided to the person; determining an indication of location of the person in relation to the guidance device or the door; and providing non-visual guidance to the person for passing through the door, wherein the non-visual guidance is based on the indication of location.

### TECHNICAL FIELD

The present disclosure relates to the field of non-visual guidance and in particular to a method, guidance device, computer program and computer program product for providing non-visual guidance for passing through a door.

### BACKGROUND

Navigating new indoor and outdoor environments is often not an easy task for people. For people with visual impairments this task becomes even more difficult. It is known to provide tactile markings on floors are to help visually-impaired people to orient themselves and move around in large spaces. However, tactile markings are not available everywhere and installation costs are significant.

It would be of great benefit if more flexible and intuitive ways are provided to allow a person to find his or her way to pass through a door.

### SUMMARY

One object is to provide an improved way to guide a person to find his or her way to pass through a door.

According to a first aspect, it is provided a method for providing non-visual guidance for passing through a door. The method is performed by a guidance device. The method comprises: determining that a person is near the door and that non-visual guidance is to be provided to the person, which comprises detecting a nearby user device, and determining that the user device is configured to indicate the need for non-visual guidance for passing through the door based on an identity of the door; determining an indication of location of the person in relation to the guidance device or the door; and providing non-visual guidance to the person for passing through the door, wherein the non-visual guidance is based on the indication of location.

The determining a nearby person may comprise detecting, in an image captured by a camera, a person with a white cane.

The determining a nearby person may comprise detecting a nearby user device and determining that the user device is configured to indicate the need for non-visual guidance for passing through the door.

The determining a nearby person comprises detecting a nearby user device and determining that the user device is configured to indicate the need for non-visual guidance for passing through the door based on the identity of the door.

The determining an indication of location may comprise using an image captured by a camera to determine a distance from the camera to the person.

The determining an indication of location may comprise determining a distance to a user device of the person using wireless ranging.

The determining an indication of location and providing non-visual guidance may be repeated until the person passes through the door.

The non-visual guidance may comprise a first modulation of a sound or vibration to indicate distance between the person and the guidance device or the door, wherein the first modulation is detectable by a human.

The first modulation may be based on a variation of at least one of a sound pitch, a sound volume, a vibration intensity, and a pulse modulation of sound or vibration.

The non-visual guidance may comprise a second modulation of a sound or vibration to indicate an orientation of the person in relation to the guidance device or the door, wherein the second modulation is detectable by a human.

The second modulation may be based on a variation of at least one of a sound pitch, a sound volume, a vibration intensity, a pulse modulation of sound and a pule modulation of vibration, and wherein the second modulation is different from the first modulation when the first modulation is present.

The providing non-visual guidance may comprise providing a peak modulation when the person is oriented towards the guidance device 1 or the door 15.

The non-visual guidance may include an indication of a door type of the door.

The non-visual guidance may include, when the door type is a revolving door, an indication of direction of rotation of the revolving door.

The non-visual guidance may include, when the door type is a sliding door or a swing door, an indication of direction of opening.

The providing non-visual guidance may comprise generating sound for output on speakers mounted near the door.

The providing non-visual guidance may comprise sending a signal to a user device of the person for generating a sound for output to the person.

The providing non-visual guidance may comprises sending a signal to a user device of the person for generating a vibration for output to the person.

According to a second aspect, it is provided a guidance device for providing non-visual guidance for passing through a door. The guidance device comprises: a processor; and a memory storing instructions that, when executed by the processor, cause the guidance device to: determine that a person is near the door and that non-visual guidance is to be provided to the person, which comprises detecting a nearby user device, and determining that the user device is configured to indicate the need for non-visual guidance for passing through the door based on an identity of the door; determine an indication of location of the person in relation to the guidance device or the door; and provide non-visual guidance to the person for passing through the door, wherein the non-visual guidance is based on the indication of location.

According to a third aspect, it is provided a computer program for providing non-visual guidance for passing through a door. The computer program comprises computer program code which, when executed on a guidance device causes the guidance device to: determine that a person is near the door and that non-visual guidance is to be provided to the person, which comprises detecting a nearby user device, and determining that the user device is configured to indicate the need for non-visual guidance for passing through the door based on an identity of the door; determine an indication of location of the person in relation to the guidance device or the door; and provide non-visual guidance to the person for passing through the door, wherein the non-visual guidance is based on the indication of location.

According to a fourth aspect, it is provided a computer program product comprising a computer program according to the third aspect and a computer readable means comprising non-transitory memory in which the computer program is stored.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIG. 1 is a schematic diagram illustrating an environment in which embodiments presented herein can be applied;

FIG. 2 is a schematic diagram illustrating an environment in which embodiments presented herein can be applied for determining distance between the guidance device and the user device;

FIGS. 3A-C are schematic diagrams illustrating various door configurations for which non-visual guidance can be provided;

FIG. 4 is a flow chart illustrating embodiments of methods for providing non-visual guidance for passing through a door;

FIG. 5 is a schematic diagram illustrating components of the guidance device of FIG. 1; and

FIG. 6 shows one example of a computer program product comprising computer readable means

### DETAILED DESCRIPTION

The aspects of the present disclosure will now be described more fully hereinafter with reference to the accompanying drawings, in which certain embodiments of the invention are shown. These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

According to embodiments presented herein, non-visual guidance is provided by a guidance device to allow a person to find the way to pass through a door. The guidance is provided based on a location of the person. The guidance device thus provides non-visual guidance to assist the person to find the door and pass through the door. The non-visual guidance can be sound and/or vibration based. In this way, the person can safely be guided to pass through the door, which can be particularly useful e.g. if the person is visually impaired.

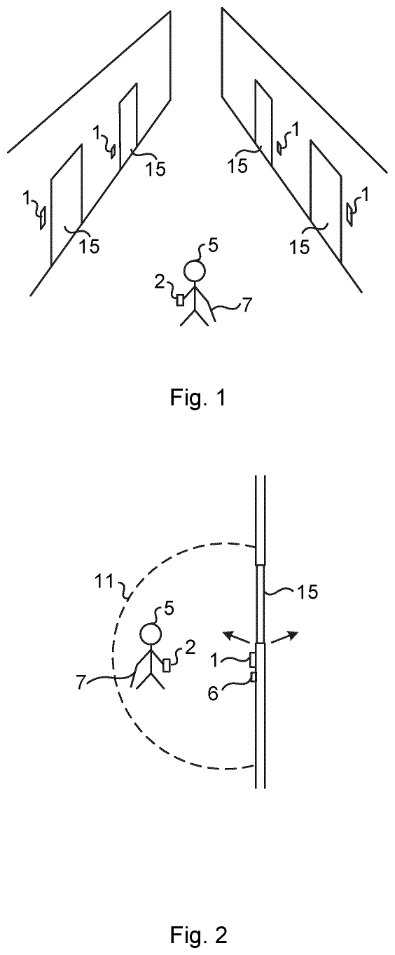

FIG. 1 is a schematic diagram illustrating an environment in which embodiments presented herein can be applied. An environment is provided in which a person 5 benefits from guidance to the correct door 15. The doors can be part e.g. of a shopping centre, public transport (for entering e.g. a train), an office, a factory or a residential home.

The person 5 optionally carries a user device 2. The user device can e.g. be a smartphone, a wearable device (smartwatch, smart jewellery, etc.) or any other portable electronic device. Optionally, the person 5 is visually impaired and carries a white cane 7. It is to be noted that the terms person and user are used interchangeably herein.

In the immediate vicinity (e.g. within a metre or two) of each door 15 that is provided according to embodiments herein, a guidance device 1 is provided. Alternatively, the processing functionality of the guidance device 1 is partly or fully provided on a remote server connected to local components (such as camera, communication modules, and/or speaker). The remote server can be connected to the local components over a wide-area network, such as the Internet. The remote server can be considered to be located in what is known as the cloud.

As explained in more detail below, each guidance device 1 is used to provide non-visual guidance to the person 5 for assisting the person to find the door 15 and pass through the door 15. Passing through the door is here to be interpreted as passing through a door opening when the door 15 is open, i.e. not passing through the door leaf of the door 15. The non-visual guidance can be in the form of sound provided using a speaker forming part of the guidance device or provided near the guidance device 1. Alternatively or additionally, the sound can be provided using the user device, based on signalling from the guidance device 1. Alternatively or additionally, the non-visual guidance can be in the form of vibrations provided using the user device 2, based on signalling from the guidance device 1. The non-visual guidance can be in the form of voice indications and/or sound signals (and/or vibrations) interpreted by the user 5 to find the door 15 in question.

FIG. 2 is a schematic diagram illustrating an environment in which embodiments presented herein can be applied for determining distance between the guidance device 1 and the user device 2.

In this embodiment, the guidance device 1 determines an indication of location of the person using wireless ranging between the guidance device 1 and the user device 2. The indication of location can then be expressed as a distance and/or a signal level (e.g. received signal strength). The wireless ranging can be achieved e.g. using Bluetooth, Bluetooth Low Energy (BLE) or Ultra-wideband (UWB). The wireless ranging allows the guidance device to determine a distance to the user device 2. This can be used to determine whether non-visual guidance is to be provided. For instance, when the user device 2 is closer than a threshold distance/threshold signal level 11, this can be used to trigger non-visual guidance to be performed. When the user device 2 is further away than the threshold distance/threshold signal level, non-visual guidance is not performed. In this way, an active zone for non-visual guidance is defined as a semicircle on the outside of the door 15. This allows multiple doors 15 with respective guidance devices 1 to function without concurrent guidance to the same person from multiple guidance devices 1.

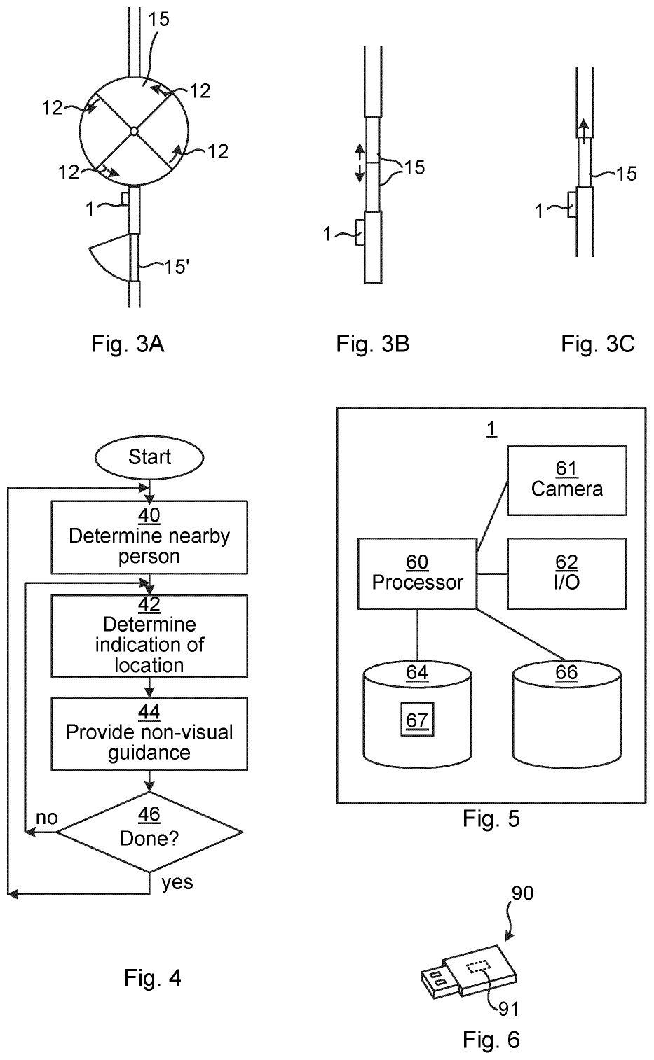

FIGS. 3A-C are schematic diagrams illustrating various door configurations for which non-visual guidance can be provided.

In FIG. 3A, it shown a door 15 in the form of a revolving door. The revolving door 15 rotates in the indicated direction 12, i.e. clockwise in this example. Optionally, a side door 15′, in the form of a swing door, is provided to make it easier for disabled or visually impaired people to pass through. In this embodiment, the non-visual guidance includes the indication of the direction 12 of rotation of the revolving door. Alternatively, when the side door 15′ is provided, the non-visual guidance guides the person to pass through the side door 15′, to thereby avoid the need to pass through the revolving door 15. For instance, the non-visual guidance can include voice synthesis mentioning that there is a revolving door but that there is a side door on the right. When a side door is not provided, the non-visual guidance can include voice synthesis mentioning that there is a revolving door that rotates clockwise.

In FIG. 3B, it shown a door 15 in the form of a sliding door 15. In this embodiment, the sliding door 15 comprises two door leaves that slide outwards away from each other to open, and slide towards each other to close. In this embodiment, the non-visual guidance can include an indication of direction of opening of the sliding door. For instance, the non-visual guidance can include voice synthesis mentioning that the door is a two-leaf sliding door.

In FIG. 3C, it shown a door 15 in the form of a sliding door 15. In this embodiment, the sliding door 15 comprises only one leaf that, in this example, slides upwards (in the view of FIG. 3C) to open, and slides downwards to close.

In this embodiment, the non-visual guidance can include an indication of direction of opening of the sliding door. For instance, the non-visual guidance can include voice synthesis mentioning that the door is a single-leaf sliding door opening to the left (from the outside).

Optionally, for a sliding door, the non-visual guidance can include an indication of the need to press a button to open the door, placement of such a button, and/or current state of the door (open or closed).

It is to be noted that also for a swing door, the non-visual guidance can optionally include an indication of direction of opening, as well as handle/doorknob placement, current state of the door (open or closed) and/or if there is any automation on the door.

FIG. 4 is a flow chart illustrating embodiments of methods for providing non-visual guidance for passing through a door. The method is performed by a guidance device 1.

In a determine nearby person step 40, the guidance device 1 determines 40 that a person 5 is near the door 15 and that non-visual guidance is to be provided to the person 5.

This step can be achieved by detecting, in an image captured by a camera, a person with a white cane.

Alternatively or additionally, this step is conditional on user configuration. This can be implemented by the guidance device 1 detecting a nearby user device 2 (e.g. using wireless communication) and its identity and determining, based on the identity, that the user device 2 is configured to indicate the need for non-visual guidance for passing through the door. This prevents inadvertent guidance for which could occur for any person passing by with a walking stick or e.g. crutches that could be mistaken for a white cane. Such configuration can be obtained using direct communication with the user device 2 or communication with a server, based on the identity of the user device 2. In other words, the user device 2 of the person is used to obtain user preferences that have been configured.

Further conditions for guidance based on user preferences can be applied. For instance, this can comprise determining that the user (person), identified by his/her user device detected by guidance device, has a preference for non-visual guidance for passing through the door also based on an identity of the door. The user preferences can indicate a preference to provide guidance for retail shops of a one or more categories (e.g. grocery stores, pharmacies, pet stores) and/or of one or more specified retail chains and/or one or more specific establishments (e.g. shops) in a respective location. In one embodiment, the user device 2 (e.g. in an application, “app”, executing in the user device) is configured to store the user preferences, which can be communicated to the guidance device 1. In one embodiment, the user has previously configured a server to store the user preferences, based on the identity of the user device. This can be achieved by the user configuring server-stored preferences using the user device 2 (e.g. in an application, “app”, executing in the user device) or a web browser on the user device 2 on any other computer. The guidance device 1 then obtains the identity of the user device 2, as described above, and queries the server to obtain the user preferences associated with the user device 2 that result in non-visual guidance being provided dependent on the identity of the door.

When the guidance device 1 is provided by a door of an establishment that matches the user preferences, the method proceeds. Otherwise, when the establishment does not match the user preferences, this step is restarted to be able to detect any new nearby person.

The term near can imply that the person is within a threshold distance from the guidance device 1 (and/or from the door 15), e.g. determined from the image. Alternatively, the term near can imply that the user device 2 is detected using wireless communication. Alternatively, near can imply that a signal level (e.g. measured as received signal strength) of a signal from the user device 2 is greater than a signal threshold.

In a determine indication of location step 42, the guidance device 1 determines an indication of location of the person 5 in relation to the guidance device 1 or the door 15. The indication of location can be a distance from the guidance device 1/door 15 to the person 5/user device 2. Optionally, the indication of location is more refined and indicates a position in a two-dimensional space of the person 5/user device 2 in relation to the door 15/guidance device 1.

The indication of location can be determined using an image captured by a camera 61 to determine a distance from the camera 61 to the person 5. The image can be a conventional two-dimensional image, a three-dimensional image or an image based on radar/lidar, etc.

Optionally, this step comprises determining a distance to a user device 2 of the person using wireless ranging.

In a provide non-visual guidance step 44, the guidance device 1 provides non-visual guidance to the person for passing through the door 15. The non-visual guidance is based on the indication of location.

The non-visual guidance can comprise a first modulation of a sound or vibration to indicate distance between the person 5 and the guidance device 1 or the door 15. The first modulation is detectable by a human. For instance, the first modulation can be based on a variation of at least one of a sound pitch, a sound volume, a vibration intensity, and/or a pulse modulation of sound or vibration.

The non-visual guidance optionally comprises a second modulation of a sound or vibration to indicate an orientation of the person in relation to the guidance device 1 or the door 15. Also the second modulation is detectable by a human. The second modulation can e.g. be based on a variation of at least one of a sound pitch, a sound volume, a vibration intensity, and/or a pulse modulation of sound and a pule modulation of vibration. The second modulation is different from the first modulation when the first modulation is present. The second modulation can be configured such that a peak modulation is provided when the person is oriented towards the guidance device 1 or the door 15. The peak can e.g. be a peak pitch or peak pulse modulation. This allows the user 5 to easily determine when the orientation is towards the guidance device 1 or the door 15.

In one embodiment, the non-visual guidance includes an indication of a door type of the door 15. For instance, the indication of door type can be that the door is a swing door, a sliding door or a revolving door. In this case, for a revolving door, the non-visual guidance optionally includes an indication of direction of rotation of the revolving door. For a sliding door or a swing door, the non-visual guidance optionally includes an indication of direction of opening.

The non-visual guidance can be in the form of sound and/or haptic vibrations. When sound-based, the guidance can contain generated sounds, such as beeps, etc., and/or can be voice based (either recorded or generated voice sounds).

When the guidance is in the form of sound, the providing the non-visual guidance can comprise generating sound for output on speakers mounted near the door 15, e.g. part of the guidance device 1 or an electronic lock by the door 15. Alternatively or additionally, the non-visual guidance is provided by sending a signal to a user device 2 of the person for (the user device 2) generating a sound for output to the user.

Alternatively or additionally, the non-visual guidance is provided by sending a signal to a user device 2 of the person for (the user device 2) generating a vibration for output to the user.

In a conditional done step 46, the guidance device determines when the guidance is finished. For instance, the guidance can be considered finished when the person 5 passes through the door 15 or if a timeout expires since the guidance was started and the person has not yet passed through the door 15. When guidance is determined to be finished, the present iteration of the method ends, and the method returns to the determine nearby person step 40 to be able to detect any new nearby person. Otherwise, when guidance is not determined to be finished, the method returns to the determine indication of location step 42.

FIG. 5 is a schematic diagram illustrating components of the guidance device 1 of FIG. 1. A processor 60 is provided using any combination of one or more of a suitable central processing unit (CPU), graphics processing unit (GPU), multiprocessor, microcontroller, digital signal processor (DSP), etc., capable of executing software instructions 67 stored in a memory 64, which can thus be a computer program product. The processor 60 could alternatively be implemented using an application specific integrated circuit (ASIC), field programmable gate array (FPGA), etc. The processor 60 can be configured to execute the method described with reference to FIG. 4 above.

The memory 64 can be any combination of random-access memory (RAM) and/or read-only memory (ROM). The memory 64 also comprises non-transitory persistent storage, which, for example, can be any single one or combination of magnetic memory, optical memory, solid-state memory or even remotely mounted memory.

A data memory 66 is also provided for reading and/or storing data during execution of software instructions in the processor 60. The data memory 66 can be any combination of RAM and/or ROM.

The guidance device 1 further comprises an I/O interface 62 for communicating with external and/or internal entities. The I/O interface 62 can e.g. include a speaker for conveying non-visual guidance in the form of sound. The I/O interface can comprise one or more communication modules for communicating with the user device 2.

A camera 61 or other image capturing device can be provided to capture an image, e.g. based on a conventional two-dimensional imaging, a three-dimensional imaging or based on radar/lidar, etc.

Other components of the guidance device 1 are omitted in order not to obscure the concepts presented herein.

FIG. 6 shows one example of a computer program product 90 comprising computer readable means. On this computer readable means, a computer program 91 can be stored in a non-transitory memory. The computer program can cause a processor to execute a method according to embodiments described herein. In this example, the computer program product is in the form of a removable solid-state memory, e.g. a Universal Serial Bus (USB) drive. As explained above, the computer program product could also be embodied in a memory of a device, such as the computer program product 64 of FIG. 5. While the computer program 91 is here schematically shown as a section of the removable solid-state memory, the computer program can be stored in any way which is suitable for the computer program product, such as another type of removable solid-state memory, or an optical disc, such as a CD (compact disc), a DVD (digital versatile disc) or a Blu-Ray disc.

It will now be presented a set of embodiments enumerated with roman numerals.

i. A method for providing non-visual guidance for passing through a door, the method being performed by a guidance device, the method comprising:
determining that a person is near the door and that non-visual guidance is to be provided to the person;
determining an indication of location of the person in relation to the guidance device or the door; and
providing non-visual guidance to the person for passing through the door, wherein the non-visual guidance is based on the indication of location.

ii. The method according to embodiment i, wherein the determining a nearby person comprises detecting, in an image captured by a camera, a person with a white cane.

iii. The method according to embodiment i or ii, wherein the determining a nearby person comprises detecting a nearby user device and determining that the user device is configured to indicate the need for non-visual guidance for passing through the door.

iv. The method according to embodiment iii, wherein the determining a nearby person comprises detecting a nearby user device and determining that the user device is configured to indicate the need for non-visual guidance for passing through the door based on an identity of the door.

V. The method according to any one of the preceding embodiments, wherein the determining an indication of location comprises using an image captured by a camera to determine a distance from the camera to the person.

vi. The method according to any one of the preceding embodiments, wherein the determining an indication of location comprises determining a distance to a user device of the person using wireless ranging.

vii. The method according to any one of the preceding embodiments, wherein the determining an indication of location and providing non-visual guidance is repeated until the person passes through the door.

viii. The method according to embodiment vii, wherein the non-visual guidance comprises a first modulation of a sound or vibration to indicate distance between the person and the guidance device or the door, wherein the first modulation is detectable by a human.

ix. The method according to embodiment viii, wherein the first modulation is based on a variation of at least one of a sound pitch, a sound volume, a vibration intensity, and a pulse modulation of sound or vibration.

X. The method according to any one of embodiments vii to ix, wherein the non-visual guidance comprises a second modulation of a sound or vibration to indicate an orientation of the person in relation to the guidance device or the door, wherein the second modulation is detectable by a human.

xi. The method according to embodiment x, wherein the second modulation is based on a variation of at least one of a sound pitch, a sound volume, a vibration intensity, a pulse modulation of sound and a pule modulation of vibration, and wherein the second modulation is different from the first modulation when the first modulation is present.

xii. The method according to embodiment x or xi, wherein the providing non-visual guidance comprises providing a peak modulation when the person is oriented towards the guidance device 1 or the door 15.

xiii. The method according to any one of the preceding embodiments, wherein the non-visual guidance includes an indication of a door type of the door.

xiv. The method according to embodiment xiii, wherein the non-visual guidance includes, when the door type is a revolving door, an indication of direction of rotation of the revolving door.

xv. The method according to embodiment xiii, wherein the non-visual guidance includes, when the door type is a sliding door or a swing door, an indication of direction of opening.

xvi. The method according to any one of the preceding embodiments, wherein the providing non-visual guidance comprises generating sound for output on speakers mounted near the door.

xvii. The method according to any one of the preceding embodiments, wherein the providing non-visual guidance comprises sending a signal to a user device of the person for generating a sound for output to the user.

xviii. The method according to any one of the preceding embodiments, wherein the providing non-visual guidance comprises sending a signal to a user device of the person for generating a vibration for output to the user.

xix. A guidance device for providing non-visual guidance for passing through a door, the guidance device comprising:
a processor; and
a memory storing instructions that, when executed by the processor, cause the guidance device to:
determine that a person is near the door and that non-visual guidance is to be provided to the person;
determine an indication of location of the person in relation to the guidance device or the door; and
provide non-visual guidance to the person for passing through the door, wherein the non-visual guidance is based on the indication of location.

XX. A computer program for providing non-visual guidance for passing through a door, the computer program comprising computer program code which, when executed on a guidance device causes the guidance device to:
determine that a person is near the door and that non-visual guidance is to be provided to the person;
determine an indication of location of the person in relation to the guidance device or the door; and
provide non-visual guidance to the person for passing through the door, wherein the non-visual guidance is based on the indication of location.

xxi. A computer program product comprising a computer program according to embodiment xx and a computer readable means comprising non-transitory memory in which the computer program is stored.

The aspects of the present disclosure have mainly been described above with reference to a few embodiments. However, as is readily appreciated by a person skilled in the art, other embodiments than the ones disclosed above are equally possible within the scope of the invention, as defined by the appended patent claims. Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-21. (canceled)

22. A method for providing non-visual guidance for passing through a door, the method being performed by a guidance device, the method comprising:
determining that a person is near the door and that non-visual guidance is to be provided to the person, which comprises detecting a nearby user device using wireless communication and determining that the user device is configured to indicate a need for the non-visual guidance for passing through the door based on an identity of the door, wherein the configuration is obtained using direct communication with the user device or in communication with a server;
determining an indication of location of the person in relation to the guidance device or the door by at least one of using an image captured by a camera to determine a distance from the camera to the person or determining a distance to the user device using wireless ranging; and
providing the non-visual guidance to the person for passing through the door, wherein the non-visual guidance is based on the indication of location, wherein the providing non-visual guidance comprises at least one of generating sound for output on speakers mounted near the door, sending a signal to the user device for generating a sound for output to the person, or sending a signal to the user device for generating a vibration for output to the person.

23. The method according to claim 22, wherein the determining that the person is near the door comprises detecting, in an image captured by the camera, a person with a white cane.

24. The method according to claim 22, wherein the determining an indication of location and providing non-visual guidance is repeated until the person passes through the door.

25. The method according to claim 24, wherein the non-visual guidance comprises a first modulation of at least one of a sound or vibration to indicate distance between the person and the guidance device or the door, wherein the first modulation is detectable by a human.

26. The method according to claim 25, wherein the first modulation is based on a variation of at least one of a sound pitch, a sound volume, a vibration intensity, a pulse modulation of sound, or a pulse modulation of vibration.

27. The method according to claim 24, wherein the non-visual guidance comprises a modulation of at least one of a sound or vibration to indicate orientation of the person in relation to the guidance device or the door, wherein the modulation is detectable by a human.

28. The method according to claim 27, wherein the modulation is based on a variation of at least one of a sound pitch, a sound volume, a vibration intensity, a pulse modulation of sound, or a pulse modulation of vibration.

29. The method according to claim 25, wherein the non-visual guidance comprises a second modulation of at least one of a sound or vibration to indicate orientation of the person in relation to the guidance device or the door, wherein the second modulation is detectable by a human.

30. The method according to claim 29, wherein the second modulation is based on a variation of at least one of a sound pitch, a sound volume, a vibration intensity, a pulse modulation of sound, or a pulse modulation of vibration, and wherein the second modulation is different from the first modulation.

31. The method according to claim 27, wherein the providing non-visual guidance comprises providing a peak modulation for the modulation when the person is oriented toward the guidance device or the door.

32. The method according to claim 22, wherein the non-visual guidance includes an indication of a door type of the door.

33. The method according to claim 32, wherein the door type is a revolving door, and wherein the non-visual guidance includes an indication of direction of rotation of the revolving door.

34. The method according to claim 32, wherein the door type is a sliding door or a swing door, and wherein the non-visual guidance includes an indication of direction of opening.

35. A guidance device for providing non-visual guidance for passing through a door, the guidance device comprising:
a processor; and
a memory storing instructions that, when executed by the processor, cause the guidance device to:
determine that a person is near the door and that non-visual guidance is to be provided to the person, which comprises detecting a nearby user device using wireless communication and determining that the user device is configured to indicate a need for the non-visual guidance for passing through the door based on an identity of the door, wherein the configuration is obtained using direct communication with the user device or in communication with a server;
determine an indication of location of the person in relation to the guidance device or the door by at least one of using an image captured by a camera to determine a distance from the camera to the person or determining a distance to the user device using wireless ranging; and
provide the non-visual guidance to the person for passing through the door, wherein the non-visual guidance is based on the indication of location, wherein the providing non-visual guidance comprises at least one of generating sound for output on speakers mounted near the door, sending a signal to the user device for generating a sound for output to the person, or sending a signal to the user device for generating a vibration for output to the person.

36. A non-transitory computer readable medium storing a computer program for providing non-visual guidance for passing through a door, the computer program comprising computer program code which, when executed on a guidance device, causes the guidance device to:
determine that a person is near the door and that non-visual guidance is to be provided to the person, which comprises detecting a nearby user device using wireless communication and determining that the user device is configured to indicate a need for the non-visual guidance for passing through the door based on an identity of the door, wherein the configuration is obtained using direct communication with the user device or in communication with a server;
determine an indication of location of the person in relation to the guidance device or the door by at least one of using an image captured by a camera to determine a distance from the camera to the person or determining a distance to the user device using wireless ranging; and
provide the non-visual guidance to the person for passing through the door, wherein the non-visual guidance is based on the indication of location, wherein the providing non-visual guidance comprises at least one of generating sound for output on speakers mounted near the door, sending a signal to the user device for generating a sound for output to the person, or sending a signal to the user device for generating a vibration for output to the person.
